(19) United States
(12) Patent Application Publication
(10) Pub. No.: US 2024/0057897 A1
(43) Pub. Date: Feb. 22, 2024

(54) NON-CONTACT HEALTH MONITORING AND PHYSIOTHERAPY EQUIPMENT, MONITORING METHOD, AND PHYSIOTHERAPY METHOD THEREOF

(71) Applicant: UNION HOSPITAL TONGJI MEDICAL COLLEGE HUST, Wuhan (CN)

(72) Inventors: Yang JIN, Wuhan (CN); Wei GENG, Wuhan (CN); Juanjuan Xu, Wuhan (CN); Yice SUN, Wuhan (CN); Xinyi ZHOU, Wuhan (CN)

(73) Assignee: UNION HOSPITAL TONGJI MEDICAL COLLEGE HUST, Wuhan (CN)

(21) Appl. No.: 18/450,488

(22) Filed: Aug. 16, 2023

(30) Foreign Application Priority Data
Aug. 22, 2022 (CN) 202211007985.1

(51) Int. Cl. A61B 5/11; A61B 5/00; A63B 22/02; A61G 7/05

(52) U.S. Cl. CPC A61B 5/1128; A61B 5/1121; A61B 5/6891; A61B 5/1116; A61B 5/6895; A63B 22/02; A61G 7/0507

(57) ABSTRACT

Some embodiments of the disclosure provide non-contact health monitoring and physiotherapy equipment. In some examples, the equipment includes a bed, a monitoring module, a physiotherapy module, a displacement device, and a machine vision monitoring device. An execution part of the displacement device is connected to the bed and/or the monitoring module to enable at least one of the bed and the monitoring module to move relative to each other. The machine vision monitoring device is mounted on the bed or the monitoring module and connected to the displacement device in a communication manner. The machine vision monitoring device is configured to identify a limb position of the patient lying on the bed to provide coordinates for moving monitoring module to the displacement device. The displacement device is configured to drive the monitoring module to align with any part of the bed from any angle.

### CROSS REFERENCE TO RELATED APPLICATIONS

This application claims priority to Chinese application number 202211007985.1, filed on Aug. 22, 2022, the disclosure of which is incorporated by reference herein in its entirety.

### FIELD OF THE DISCLOSURE

The disclosure relates generally to the field of measuring instruments for diagnostic purposes. More specifically, the disclosure relates to the field of non-contact health monitoring and physiotherapy equipment.

### BACKGROUND

In the field of medical monitoring, there is no suitable method for automatically monitoring a burn patient. At present, the burn patient is mainly patrolled and monitored by medical staff.

Because skin of the burn patient has been damaged, a monitoring head of contact health monitoring equipment easily causes injuries to the burn patient, while non-contact health monitoring equipment that can monitor a whole body is bulky and usually arranged in different departments. This is impossible to monitor key indicators of the burn patient.

At present, related technologies disclose design concepts of equipment with a plurality of monitoring functions. For example, CN111938604A discloses a system for remote monitoring of respiratory diseases based on a multi-modal technology. However, there are still many problems to be resolved when the design concepts are actually used. A working distance of near infrared imaging and a working distance of far infrared imaging are different, videoing of visible light imaging requires a long distance, and microphone monitoring requires a close distance. Therefore, a technical problem of how to monitor a health status of the patient may not be resolved by simply integrating a plurality of functions into one piece of equipment.

In addition, monitoring alone cannot relieve pains of the patient, and sometimes corresponding physiotherapy services are necessary to be provided. However, due to space limitations, there is no space for physiotherapy equipment after monitoring equipment is arranged around a hospital bed.

Therefore, a problem to be resolved by persons is how to overcome defects of a conventional technology and integrate a plurality pieces of non-contact monitoring equipment and physiotherapy equipment, to monitor a plurality of key indicators of one patient and provide physiotherapy services at the same time.

### SUMMARY

The following presents a simplified summary of the invention in order to provide a basic understanding of some aspects of the invention. This summary is not an extensive overview of the invention. It is not intended to identify critical elements or to delineate the scope of the invention. Its sole purpose is to present some concepts of the invention in a simplified form as a prelude to the more detailed description that is presented elsewhere.

In some embodiments, the disclosure provide a non-contact health monitoring and physiotherapy equipment which includes: a bed; a monitoring module, where the monitoring module is configured to: obtain vital signs of a part of a human body of a patient in real time in a non-contact manner, and output the vital signs by a human-computer interaction terminal; a physiotherapy module, integrally mounted on the monitoring module, where the physiotherapy module is configured to provide a non-contact physiotherapy service to the patient; a displacement device, where an execution part of the displacement device is connected to the bed and/or the monitoring module, to enable at least one of the bed and the monitoring module to move relative to each other, and the displacement device has a moving range in which the monitoring module is aligned with any part of the bed from any angle; and a machine vision monitoring device, where the machine vision monitoring device is mounted on the bed or the monitoring module and connected to the displacement device in a communication manner, and the machine vision monitoring device is configured to identify a limb position of the patient lying on the bed, to provide the displacement device with coordinates for moving the monitoring module.

Further, the displacement device includes: a walking device, capable of moving on a ground, a wall, or a ceiling; a linear driver, connected to an execution part of the walking device, where a driving direction of the linear driver is vertical; and an angular displacement device, connected to an execution part of the linear driver, where the angular displacement device has at least one degree of freedom, and the execution part of the angular displacement device is connected to the monitoring module.

Further, the displacement device includes a rail and a rail vehicle, where the rail is mounted on a ground, a wall, or a ceiling and suspended above the patient, the rail vehicle is connected to the rail in a sliding manner, and the monitoring module is connected to the rail vehicle. The rail includes: a first rail, disposed horizontally; a second rail, where one end of the second rail is rotationally connected to the first rail; and a telescopic rod, where two ends of the telescopic rod are respectively connected to free ends of the first rail and the second rail.

Further, the rail further includes a first limiting plate and a second limiting plate that are respectively disposed above the first rail and the second rail. The first rail is parallel to the first limiting plate, the second rail is parallel to the second limiting plate, and adjacent ends of the first limiting plate and the second limiting plate are hinged.

Further, the one end of the second rail is hinged with one end of the second limiting plate by a first connecting rod, and the other end of the second rail is hinged with the other end of the second limiting plate by a second connecting rod.

Further, the rail vehicle includes: a first rotating part, disposed at one end of the rail; a second rotating part, disposed at the other end of the rail; a flexible strip, where two ends of the flexible strip are wound and frictionally connected to the first rotating part and the second rotating part; a driver, where an execution part of the driver is connected to the first rotating part and/or the second rotating part, and the driver is configured to drive the first rotating part and the second rotating part to rotate, to enable one of the first rotating part and the second rotating part to wrap the flexible strip and enable the other one of the first rotating part and the second rotating part to release the flexible strip; a vehicle body, connected to a middle part of the flexible strip; and wheels, disposed on the rail and rotationally connected to the vehicle body.

Further, the vehicle body includes: at least two sliding rods, disposed side by side, where the sliding rods are perpendicular to the rail, and the wheels are rotationally connected to two ends of the sliding rods; a connector, vertically connected to the sliding rods to form a rectangular frame; a slider, connected to the sliding rods in a sliding manner and fastened to the monitoring module; a rack, formed on one of the sliding rods and extending along a length direction of the sliding rod; a gear, rotationally connected to the slider and meshed with the rack; and a second motor, connected to the slider, where an execution shaft is coaxially connected to the gear.

Further, the monitoring module further includes an angle detector. The angle detector is connected to the displacement device in a communication manner, and the angle detector is configured to detect an elevation of a headboard. The angle detector includes at least two distance sensors, and detection ends of the distance sensors are disposed upward and respectively aligned with two parts of the headboard with different heights.

In other embodiments, the disclosure provides a monitoring method of non-contact health monitoring and physiotherapy equipment which includes: obtaining a limb position of a patient on a bed by a machine vision monitoring device; obtaining vital signs of a partial body of the patient by a monitoring module in a non-contact manner; moving the bed or the monitoring module, to enable the monitoring module to obtain vital signs of a whole body of the patient; and outputting the vital signs of the whole body of the patient by a human-computer interaction terminal.

In further embodiments, the disclosure provides a physiotherapy method of non-contact health monitoring and physiotherapy equipment which includes: obtaining a limb position of a patient on a bed by a machine vision monitoring device; obtaining vital signs of a partial body of the patient by a monitoring module in a non-contact manner; moving the bed or the monitoring module, to enable the monitoring module to obtain vital signs of a whole body of the patient; and providing a non-contact physiotherapy service to the part of the patient with an abnormal vital sign by the physiotherapy module.

### BRIEF DESCRIPTION OF THE DRAWINGS

Illustrative embodiments of the present disclosure are described in detail below with reference to the attached drawing figures.

FIG. 1 is a stereoscopic view according to an embodiment of the disclosure.

FIG. 2 is a stereoscopic view and a partial enlarged view thereof according to another embodiment of the disclosure.

FIG. 3 is a top view according to another embodiment of the disclosure.

FIG. 4 is the A-A view of FIG. 3.

FIG. 5 is an exploded perspective view and a partial enlarged view thereof according to an embodiment of the disclosure.

FIG. 6 is an exploded perspective view of a partial structure of a rail vehicle according to an embodiment of the disclosure.

### DETAILED DESCRIPTION

The following describes some non-limiting exemplary embodiments of the invention with reference to the accompanying drawings. The described embodiments are merely a part rather than all of the embodiments of the invention. All other embodiments obtained by a person of ordinary skill in the art based on the embodiments of the disclosure shall fall within the scope of the disclosure.

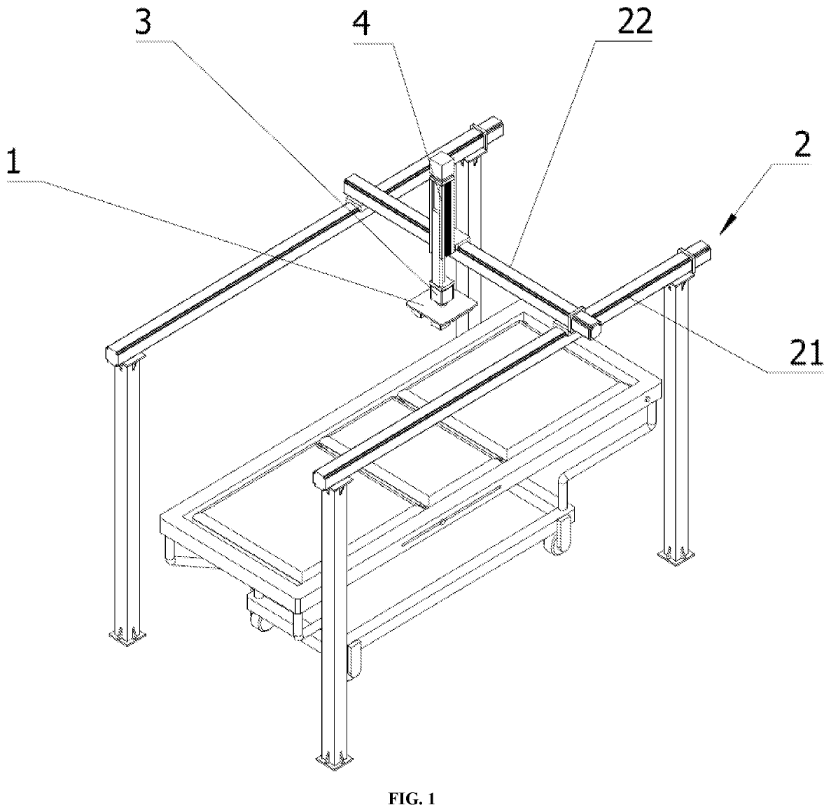

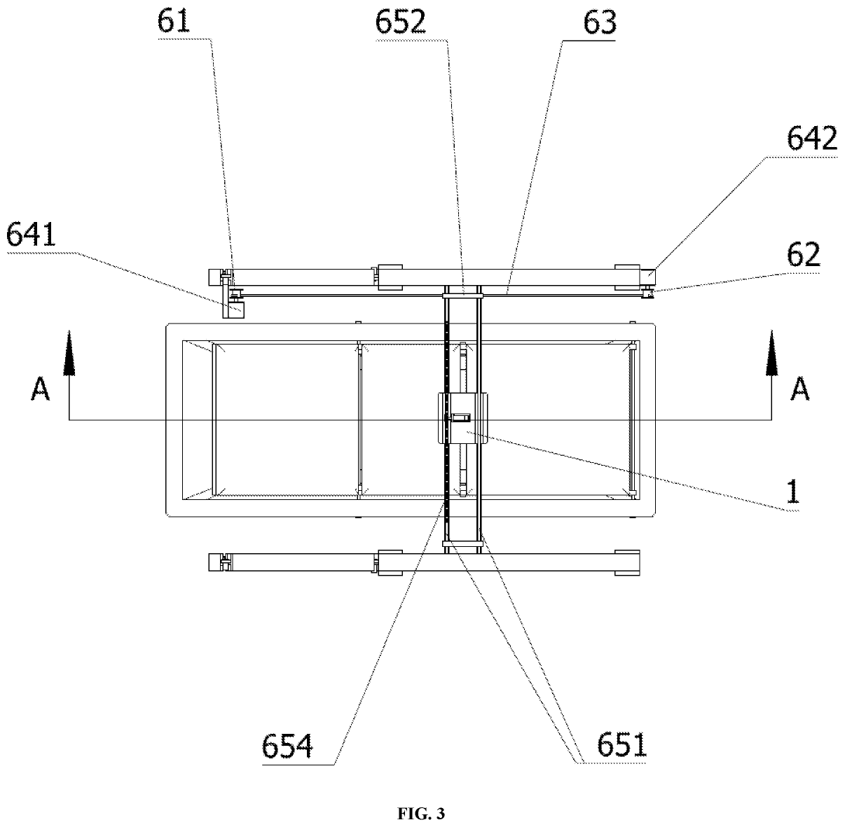

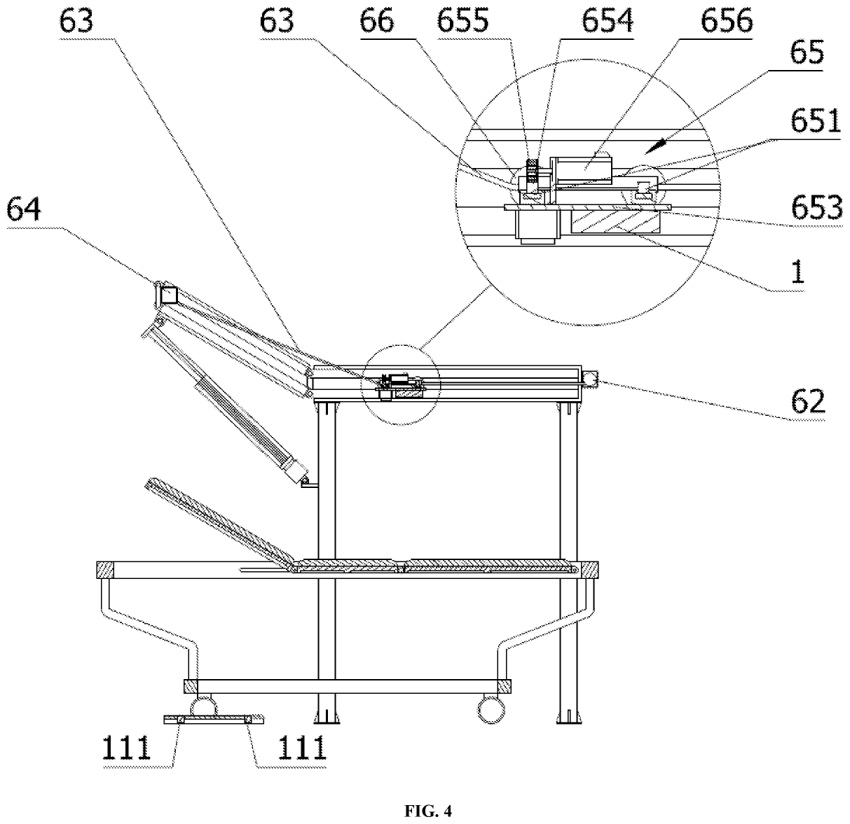

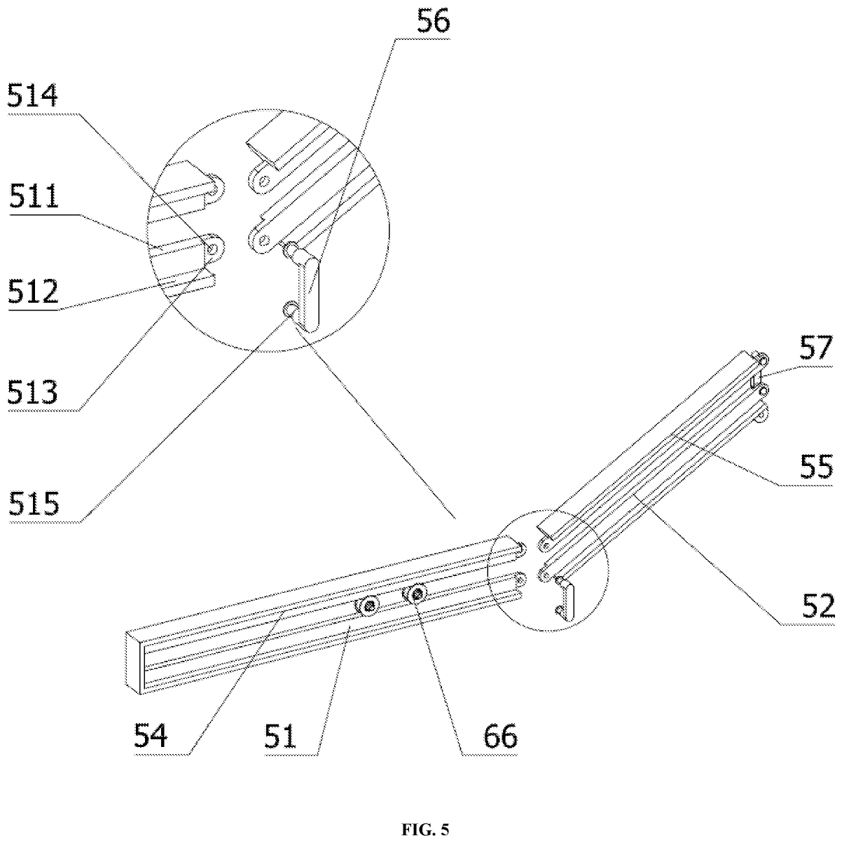

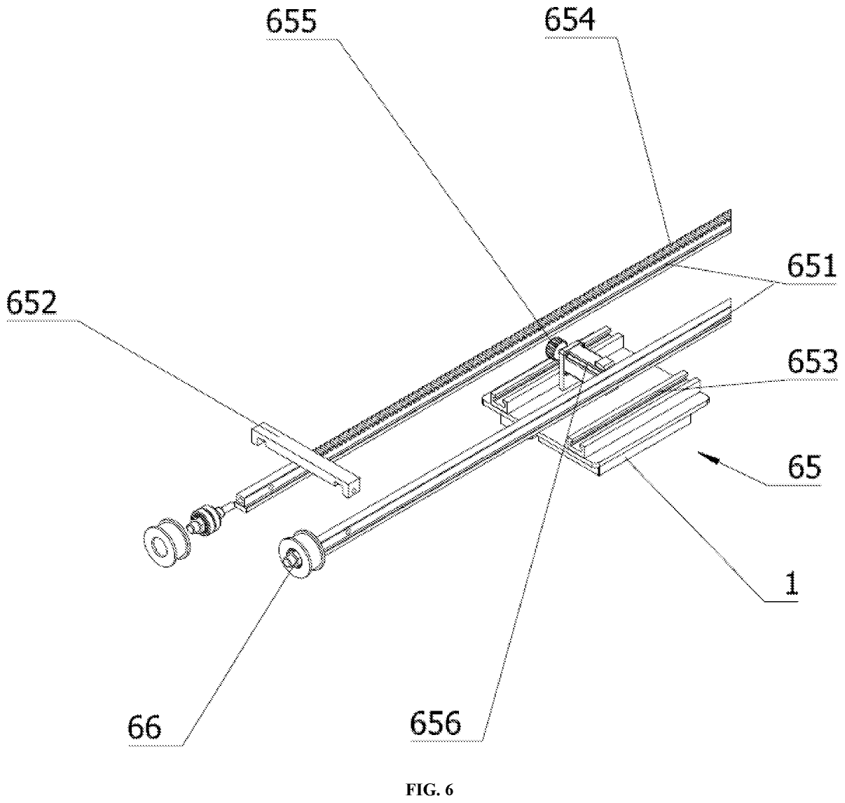

In FIGS. 1 through 6: 1 represents monitoring module, 11 represents angle detector, 111 represents distance sensor, 2 represents walking device, 21 represents first electric sliding table, 22 represents second electric sliding table, 3 represents angular displacement device, 4 represents linear driver, 5 represents rail, 51 represents first rail, 511 represents vertical plate, 512 represents transverse plate, 513 represents hinging joint, 514 represents hinging hole, 515 represents pin, 52 represents second rail, 53 represents telescopic rod, 54 represents first limiting plate, 55 represents second limiting plate, 56 represents first connecting rod, 57 represents second connecting rod, 6 represents rail vehicle, 61 represents first rotating part, 62 represents second rotating part, 63 represents flexible strip, 64 represents driver, 641 represents first motor, 642 represents coil spring, 65 represents vehicle body, 651 represents sliding rods, 652 represents connector, 653 represents slider, 654 represents rack, 655 represents gear, 656 represents second motor, and 66 represents wheels.

FIG. 1 shows an example of a non-contact health monitoring and physiotherapy equipment according to the disclosure. In this example, the equipment may include: a bed, a monitoring module 1, a physiotherapy module, a displacement device, and a machine vision monitoring device.

The monitoring module 1 is configured to: obtain vital signs of a part of a human body of a patient in real time in a non-contact manner, and output the vital signs by a human-computer interaction terminal.

The physiotherapy module is integrally mounted on the monitoring module 1, and the physiotherapy module is configured to provide a non-contact physiotherapy service to the patient.

An execution part of the displacement device is connected to the bed and/or the monitoring module 1, to enable at least one of the bed and the monitoring module 1 to move relative to each other. The displacement device has a moving range in which the monitoring module 1 is aligned with any part of the bed from any angle.

The machine vision monitoring device is mounted on the bed or the monitoring module 1 and connected to the displacement device in a communication manner, and the machine vision monitoring device is configured to identify a limb position of the patient lying on the bed, to provide the displacement device with coordinates for moving the monitoring module 1.

In the related art, a monitoring device capable of obtaining various vital signs of a whole body of a patient is bulky, but a monitoring device capable of obtaining only vital signs of a part of a human body of a patient is small. Probes of various monitoring devices may be integrated together to form the monitoring module 1.

The machine vision monitoring device recognizes an image of the body of the patient by a charge coupled device (CCD) camera and feeds the image back to the displacement device, and the displacement device moves to adjust a spatial position of the monitoring module 1, so that the monitoring module 1 may circularly move to align with a head, a chest, an abdomen, and a limb of the patient.

Due to the displacement device, the monitoring module 1 circularly moves to continuously aim at different parts of the body of the patient from different angles, to obtain key indicators. An industrial computer splices and integrates the key indicators to form key indicators of the whole body of the patient, and then outputs the key indicators of the whole body by the human-computer interaction terminal.

The monitoring module 1 may include one or a combination of a thermal imaging monitoring terminal, a microwave radar monitoring terminal, and an acoustic monitoring terminal. The thermal imaging monitoring terminal, the microwave radar monitoring terminal, and the acoustic monitoring terminal are connected to the human-computer interaction terminal in a commutation manner.

A monitoring range of the monitoring module 1 is a partial body of the patient and depends on driving of the displacement device, so that monitoring results of the monitoring module 1 may be spliced into a report of the key indicators of the whole body of the patient.

The specific number of thermal imaging monitoring terminal, the machine vision monitoring terminal, and the microwave radar monitoring terminal are not limited, for example, a combination of a plurality of thermal imaging monitoring terminals and one machine vision monitoring terminal. The plurality of thermal imaging monitoring terminals may include one or two of near infrared imaging and far infrared imaging.

It should also be noted that a working path, a moving speed, and a number of cycles of the displacement device may be manually set and automatically executed, so as to realize circulation monitoring in different periods, or select the key indicators to monitor based on conditions of the patient.

An operation module is disposed on the bed for patients or medical staff to operate, for example, adjustment of a height or an angle of the bed, to achieve the best monitoring.

The human-computer interaction terminal is not shown in the figure. However, a health status and an abnormal situation or a system error of the patient may be expressed to a medical front desk by any means such as sound, light, printing, short messages, and notification of an application (APP).

The thermal imaging monitoring terminal collects infrared radiation of a human body by a thermal infrared imager, to form a false color heat map, judge a location, nature, and a degree of a lesion of the human body, and provide a reliable basis for clinical diagnosis. A principle is that when diseases or functions of a part of the human body are changed, a blood flow and a temperature herein accordingly change, resulting that a local temperature of the human body changes.

The microwave radar monitoring terminal monitors a fluctuation of skin on a chest, and a biological radar may obtain heart and lung signals of a target, and sends a warning when a sign is abnormal. A principle is to obtain a target signal based on the fluctuation of the skin on the surface.

The acoustic monitoring terminal obtains a cough sound of the patient by a microphone, to judge whether there is effusion in a lung and trachea of the patient.

The physiotherapy module (not shown in the figure) may include a red and blue light emitting end. The red and blue light emitting end emits blue light with a wavelength of about 415 nm and red light with a wavelength of about 630 nm by a light-emitting diode (LED) chip, to treat diseases such as ulceration, phlebitis, and acne.

The blue light with a wavelength of about 415 nm has effect of quickly inhibiting inflammation. Endogenous porphyrin produced by Propionibacterium acnes absorbs the blue light with a wavelength of about 415 nm. This causes light excitation, resulting in death of bacteria. Without damage to skin tissues, such bacteria are efficiently destroyed, so that formation of acnes is reduced to the maximum extent, and acnes in inflammatory period are obviously reduced and healed in a short time.

The red light with a wavelength about 630 nm has characteristics of a high purity, a strong light source, and a uniform energy density. This stimulates fibroblasts in activated cells to produce growth factors, which may improve cell activity, and may promote metabolism of cells, so that skin secretes a large amount of collagen and fibrous tissue to fill the skin and collagen tissue regeneration is promoted, to reduce formation of scars, accelerate blood circulation, and increase skin elasticity.

Further, in an optional embodiment of this application, an example is used to describe how the displacement device drives the monitoring module 1 to align with any part of the bed from any angle.

Further, the displacement device may include: a walking device 2, a linear driver 4, an angular displacement device 3. The walking device 2 is capable of moving on a ground, a wall, or a ceiling.

The linear driver 4 is connected to an execution part of the walking device 2, and a driving direction of the linear driver 4 is vertical.

The angular displacement device 3 is connected to an execution part of the linear driver 4, the angular displacement device 3 has at least one degree of freedom, and the execution part of the angular displacement device 3 is connected to the monitoring module 1.

The walking device 2 may walk on the ground, the wall, and the ceiling through the rail, or may walk on the ground through electric universal wheels.

In this embodiment, the walking device 2 is a cartesian coordinate robot, which may include a first electric sliding table 21 and a second electric sliding table 22 that are disposed along an X-axis direction and a Y-axis direction. The angular displacement device 3 is an electric tripod, and the linear driver 4 is an electric push rod disposed along a Z-axis direction.

The electric sliding table and the electric push rod may be one or a combination of a ball screw sliding table, a rack and pinion sliding table, and a synchronous belt sliding table.

The angular displacement device 3 may include at least one rotary driver with a servo motor as a main body. An angular displacement device 3 with 2-degrees of freedom optionally may include two rotary drivers, to enable the monitoring module 1 to vertically monitor downwards limbs of the patient when the patient lies flat.

Alternatively, when the patient is half lying, the monitoring module 1 monitors chest and abdomen of the patient at a 45° downward inclination.

Alternatively, when the patient lies on side, the monitoring module 1 horizontally monitors chest and back of the patient.

Alternatively, when a temporary bed perpendicular to another bed in a ward, the monitoring module 1 rotates 90° around a vertical axis to accommodate the extra bed.

The linear driver 4 is configured to drive the angular displacement device 3, to drive the monitoring module 1 to move along an vertical track and change a height of the monitoring module 1, so that the monitoring module 1 may be aligned with the patient after an angle of the monitoring module 1 is adjusted by the angular displacement device 3.

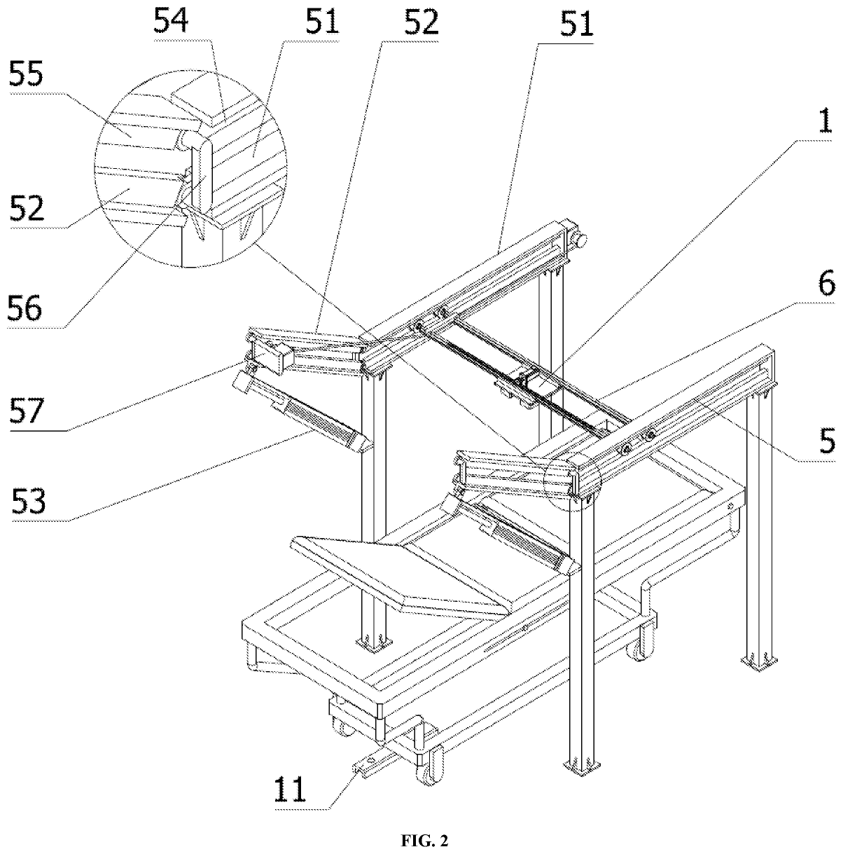

Further, a sound generated by repeated movement of the displacement device may affect normal rest of the patient. To resolve this problem, the disclosure may further provide a displacement device capable of working at a low volume, with optional embodiments as shown in FIG. 2 to FIG. 6. The displacement device is used to resolve a technical problem that a sound is louder when the displacement device frequently adjusts a horizontal position, a height, and an elevation of the monitoring module 1 at present.

The displacement device may include a rail 5 and a rail vehicle 6. The rail 5 is separately mounted on the ground, the wall, or the ceiling through an upright, a wallboard, and a hanging post, and suspended above the patient. The rail vehicle 6 is connected to the rail 5 in a sliding manner, and the monitoring module 1 is connected to the rail vehicle 6.

The rail 5 may include: a first rail 51, a second rail 52, and a telescopic rod 53. The first rail 51 is disposed horizontally.

One end of the second rail 52 is rotationally connected to the first rail 51.

Two ends of the telescopic rod 53 are respectively connected to free ends of the first rail 51 and the second rail 52.

An angle of the second rail 52 relative to a horizontal plane may be changed by elongating or shortening the telescopic rod 53, so that the second rail 52 may be flush with an angle of a head of the bed. When the head of a monitoring bed raises and the patient is in a half-laying posture, the second rail 52 also raises, so that when the rail vehicle 6 moves along the rail 5, the monitoring module 1 and the body of the patient may be vertical and a distance between the monitoring module 1 and the body of the patient is constant.

In this embodiment, the distance and an angle between the monitoring module 1 and the patient in the half-lying posture may be corrected by adjusting the rail 5 once, without repeated operation of the angular displacement device 3 and the linear driver 4. Therefore, working steps are few, so that working efficiency is high and noises are low.

In this embodiment, the first rail 51 may include a vertical plate 511 and a transverse plate 512 that are connected in an inverted T shape. The vertical plate 511 is configured to connect the rail vehicle 6 in a sliding manner, and the transverse plate 512 is configured to strengthen a strength of the vertical plate 511. A hinge head 513 of which thickness is half that of the vertical plate 511 is formed on one end of the vertical plate 511, and a hinge hole 514 is formed on the hinge head 513. A structure of the second rail 52 is the same as that of the first rail 51. The first rail 51 and the second rail 52 are rotationally connected by pins 515 passing through the hinge hole 514, and wall surfaces of the first rail 51 and the second rail 52 are flush with each other.

The first rail 51 is fastened to the ground by two uprights. The telescopic rod 53 is an electric push rod, and two ends of the telescopic rod 53 are respectively connected to the free end of the second rail 52 and the middle part of the upright through hinges.

Further, in an optional embodiment of this application, a technical problem that medical staff or family members may collide with the rail vehicle 6, resulting that the rail vehicle 6 falls off the rail 5.

The rail 5 further may include a first limiting plate 54 and a second limiting plate 55 that are respectively disposed above the first rail 51 and the second rail 52. The first rail 51 is parallel to the first limiting plate 54, the second rail 52 is parallel to the second limiting plate 55, and adjacent ends of the first limiting plate 54 and the second limiting plate 55 are hinged.

Specific shapes of the first limiting plate 54 and the second limiting plate 55 are disposed based on a specific structure of the rail vehicle 6. Normally, the rail vehicle 6 is connected to the first rail 51 and the second rail 52 by wheels 66 having an H-shaped cross section. In this case, the first limiting plate 54 and the second limiting plate 55 are symmetrical to the first rail 51 and the second rail 52.

In an optional embodiment of this application, the second limiting plate 55 and the second rail 52 synchronously move and may be parallel to each other.

The one end of the second rail 52 is hinged with one end of the second limiting plate 55 by a first connecting rod 56, and the other end of the second rail 52 is hinged with the other end of the second limiting plate 55 by a second connecting rod 57.

The first rail 51 and the first limiting plate 54 are fastened to a first connecting rod 56 by the vertical plate. The second rail 52, the second limiting plate 55, the first connecting rod 56, and a second connecting rod 57 are combined into a quadrilateral connecting rod mechanism, so that when the one end of the second rail 52 rotates, the second limiting plate 55 may synchronously move parallel to the second rail 52.

In an optional embodiment of this application, a technical problem of how the rail vehicle 6 walks on the first rail 51 and the second rail 52 is resolved.

The rail vehicle 6 may include a first rotating part 61, a second rotating part 62, a flexible strip 63, a driver 64, a vehicle body 65, and wheels 66. The first rotating part 61 is disposed at one end of the rail 5.

The second rotating part 62 is disposed at the other end of the rail 5.

Two ends of the flexible strip 63 are wound and frictionally connected to the first rotating part 61 and the second rotating part 62.

An execution part of the driver 64 is connected to the first rotating part 61 and/or the second rotating part 62, and the driver 64 is configured to drive the first rotating part 61 and the second rotating part 62 to rotate, to enable one of the first rotating part 61 and the second rotating part 62 to wrap the flexible strip 63 and enable the other one of the first rotating part 61 and the second rotating part 62 to release the flexible strip 63.

The vehicle body 65 is connected to a middle part of the flexible strip 63.

The wheels 66 are disposed on the rail 5 and rotationally connected to the vehicle body 65.

In this embodiment, the first rotating part 61 and the second rotating part 62 are both winches, and the flexible strip 63 is a steel cable. The first rotating part 61 is connected to the first motor 641, and the second rotating part 62 is connected to a coil spring 642. The first rotating part 61 is powered on to pull the rail vehicle 6 to move toward the second rail 52, and the first rotating part 61 is powered off to pull the rail vehicle 6 to move toward the first rotating part 61 through resilient force of the coil spring. A position of the vehicle body 65 on the rail 5 may be calculated based on a length of taking up or paying off the steel cable by the first rotating part 61.

The first rotating part 61 and the second rotating part 62 may also be a pulley or a chain wheel, and the flexible strip 63 may be a synchronous belt or a chain. The foregoing functions may be achieved by adding a tensioning wheel to tighten the pulley and the chain.

The cross sections of the wheels 66 are H-shaped, and may prevent derailment of the wheels 66.

The wheels 66 may also be in a common wheel shape. The wheels overlap on a top surface of the rail 5 and clamp side surfaces of the rail 5 by two auxiliary wheels, to prevent the derailment of the wheels 66.

In an optional embodiment of this application, a technical problem that the monitoring module 1 may only move in one direction along with the vehicle body 65, cannot laterally move relative to a hospital bed, and cannot move between a plurality of hospital beds is resolved.

The vehicle body 65 may include: at least two sliding rods 651, a connector 652, a slider 653, a rack 654, a gear 655, and a second motor 656. The at least two sliding rods 651 are disposed side by side, the sliding rods 651 are perpendicular to the rail 5, and the wheels 66 are rotationally connected to two ends of the sliding rods 651.

The connector 652 is vertically connected to the sliding rods 651 to form a rectangular frame.

The slider 653 is connected to the sliding rods 651 in a sliding manner and fastened to the monitoring module 1.

The rack 654 is formed on one of the sliding rods 651 and extending along a length direction of the sliding rod 651.

The gear 655 is rotationally connected to the slider 653 and meshed with the rack 654.

The second motor 656 is connected to the slider 653, where an execution shaft is coaxially connected to the gear 655.

The sliding rods 651 are in an inverted T shape. Horizontal portions of the sliding rods 651 are configured to connect the slider 653, vertical portions of the sliding rods 651 are configured to strengthen strengths of the horizontal portions of the sliding rods 651 and form the rack 654. The second motor 656 drives the gear 655 to rotate, so that the slider 653 may move along the sliding rods 651, to enable the monitoring module 1 to move along the X-axis direction and the Y-axis direction through the rail 5 and the sliding rods 651.

In an optional embodiment of this application, a technical problem that the elevation of the headboard cannot be well measured only by the monitoring module 1.

The monitoring module 1 further may include an angle detector 11. The angle detector 11 is connected to the displacement device in a communication manner, and the angle detector 11 is configured to detect an elevation of a headboard.

The angle detector 11 may include at least two distance sensors 111. Detection ends of the distance sensors 111 are disposed upward and respectively aligned with two parts of the headboard with different heights.

The angle detector 11 may be an angle sensor mounted on the headboard, or may be two distance sensors 111. The elevation of the headboard may be calculated by a trigonometric function based on a distance between the two distance sensors 111 and a height difference between the two parts of the headboard with different heights, so that the angle detector 11 may detect the elevation of the headboard without contact, and the angular displacement device 3 may be rotated to a suitable angle, or the second rail 52 may be swung to a suitable angle.

A monitoring method of non-contact health monitoring and physiotherapy equipment may include: obtaining a limb position of a patient on a bed by a machine vision monitoring device; obtaining vital signs of a partial body of the patient by a monitoring module in a non-contact manner; moving the bed or the monitoring module, to enable the monitoring module to obtain vital signs of a whole body of the patient; and outputting the vital signs of the whole body of the patient by a human-computer interaction terminal.

A physiotherapy method of non-contact health monitoring and physiotherapy equipment may include: obtaining a limb position of a patient on a bed by a machine vision monitoring device; obtaining vital signs of a partial body of the patient by a monitoring module in a non-contact manner; moving the bed or the monitoring module, to enable the monitoring module to obtain vital signs of a whole body of the patient; and providing a non-contact physiotherapy service to the part of the patient with an abnormal vital sign by the physiotherapy module.

The above examples are only exemplary in the present disclosure, and not intended to limit the present disclosure, and the protection scope of the present disclosure is defined by the claims. A person skilled in the art may make various modifications or equivalents to the present disclosure within the spirit and scope of the present disclosure. All these modifications or equivalents shall be considered to fall within the protection scope of the technical solutions of the present disclosure.

Various embodiments of the disclosure may have one or more of the following effects. In some embodiments, the disclosure may monitor various vital signs of a whole body of a patient and provide a physiotherapy service. In other embodiments, the disclosure provides a non-contact health monitoring and physiotherapy equipment, a monitoring method, and a physiotherapy method thereof, which may help to resolve a technical problem that existing non-contact monitoring equipment does not directly obtain a plurality of key indexes of a whole body of a patient. In further embodiment, the present disclosure, when compared with a conventional technology, may have the following effects. The present disclosure may provide non-contact health monitoring and physiotherapy equipment. The equipment may include a bed, a monitoring module, a physiotherapy module, a displacement device, and a machine vision monitoring device. An execution part of the displacement device may be connected to the bed and/or the monitoring module to enable at least one of the bed and the monitoring module to move relative to each other. The machine vision monitoring device may be mounted on the bed or the monitoring module and connected to the displacement device in a communication manner. The machine vision monitoring device may be configured to identify a limb position of the patient lying on the bed to provide coordinates for moving monitoring module to the displacement device. The displacement device may be configured to drive the monitoring module to align with any part of the bed from any angle, thereby obtaining vital signs of a partial body of the patient in real time in a non-contact manner and outputting vital signs of a whole body of the patient by a human-computer interaction terminal. In addition, a physiotherapy service may be provided to a part of the patient with abnormal vital signs by the physiotherapy module.

Many different arrangements of the various components depicted, as well as components not shown, are possible without departing from the spirit and scope of the present disclosure. Embodiments of the present disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

It will be understood that certain features and subcombinations are of utility and may be employed without reference to other features and subcombinations and are contemplated within the scope of the claims. Unless indicated otherwise, not all steps listed in the various figures need be carried out in the specific order described.

The disclosure claimed is:

1. A non-contact health monitoring and physiotherapy equipment, comprising:
a bed;
a monitoring module (1), wherein the monitoring module (1) is configured to obtain vital signs of a part of a human body of a patient in real time in a non-contact manner and to output the vital signs by a human-computer interaction terminal;
a physiotherapy module integrally mounted on the monitoring module (1), wherein the physiotherapy module is configured to provide a non-contact physiotherapy service to the patient;
a displacement device, wherein:
an execution part of the displacement device is connected to at least one of the bed and the monitoring module (1) to enable at least one of the bed and the monitoring module (1) to move relative to each other, and
the displacement device has a moving range in which the monitoring module (1) is aligned with any part of the bed from any angle; and
a machine vision monitoring device, wherein:
the machine vision monitoring device is mounted on the bed or the monitoring module (1) and connected to the displacement device in a communication manner, and
the machine vision monitoring device is configured to identify a limb position of the patient lying on the bed to provide the displacement device with coordinates for moving the monitoring module (1).

2. The non-contact health monitoring and physiotherapy equipment according to claim 1, wherein the displacement device comprises:
a walking device (2) configured to move on a ground, a wall, or a ceiling;
a linear driver (4) connected to an execution part of the walking device (2), wherein a driving direction of the linear driver (4) is vertical; and
an angular displacement device (3) connected to an execution part of the linear driver (4), wherein the angular displacement device (3) has at least one degree of freedom and the execution part of the angular displacement device (3) is connected to the monitoring module (1).

3. The non-contact health monitoring and physiotherapy equipment according to claim 2, wherein:
the monitoring module (1) further comprises an angle detector (11), the angle detector (11) being connected to the displacement device in a communication manner and the angle detector (11) being configured to detect an elevation of a headboard;
the angle detector (11) comprises at least two distance sensors (111); and
detection ends of the distance sensors (111) are disposed upward and respectively aligned with two parts of the headboard with different heights.

4. The non-contact health monitoring and physiotherapy equipment according to claim 1, wherein:
the displacement device comprises a rail (5) and a rail vehicle (6), wherein:
the rail (5) is mounted on a ground, a wall, or a ceiling and is suspended above the patient,
the rail vehicle (6) is connected to the rail (5) in a sliding manner, and
the monitoring module (1) is connected to the rail vehicle (6); and
the rail (5) comprises:
a first rail (51) disposed horizontally;
a second rail (52), wherein one end of the second rail (52) is rotationally connected to the first rail (51); and
a telescopic rod (53), wherein two ends of the telescopic rod (53) are respectively connected to free ends of the first rail (51) and the second rail (52).

5. The non-contact health monitoring and physiotherapy equipment according to claim 4, wherein:
the rail (5) further comprises a first limiting plate (54) and a second limiting plate (55) that are respectively disposed above the first rail (51) and the second rail (52);
the first rail (51) is parallel to the first limiting plate (54);
the second rail (52) is parallel to the second limiting plate (55); and
adjacent ends of the first limiting plate (54) and the second limiting plate (55) are hinged.

6. The non-contact health monitoring and physiotherapy equipment according to claim 5, wherein:
one end of the second rail (52) is hinged with one end of the second limiting plate (55) by a first connecting rod (56); and
another end of the second rail (52) is hinged with another end of the second limiting plate (55) by a second connecting rod (57).

7. The non-contact health monitoring and physiotherapy equipment according to claim 6, wherein:
the monitoring module (1) further comprises an angle detector (11), the angle detector (11) being connected to the displacement device in a communication manner and the angle detector (11) being configured to detect an elevation of a headboard;
the angle detector (11) comprises at least two distance sensors (111); and
detection ends of the distance sensors (111) are disposed upward and respectively aligned with two parts of the headboard with different heights.

8. The non-contact health monitoring and physiotherapy equipment according to claim 5, wherein:
the monitoring module (1) further comprises an angle detector (11), the angle detector (11) being connected to the displacement device in a communication manner and the angle detector (11) being configured to detect an elevation of a headboard;
the angle detector (11) comprises at least two distance sensors (111); and
detection ends of the distance sensors (111) are disposed upward and respectively aligned with two parts of the headboard with different heights.

9. The non-contact health monitoring and physiotherapy equipment according to claim 4, wherein the rail vehicle (6) comprises:
a first rotating part (61) disposed at one end of the rail (5);
a second rotating part (62) disposed at another end of the rail (5);
a flexible strip (63), wherein two ends of the flexible strip (63) are wound and frictionally connected to the first rotating part (61) and the second rotating part (62);
a driver (64), wherein:
an execution part of the driver (64) is connected to at least one of the first rotating part (61) and the second rotating part (62), and
the driver (64) is configured to drive the first rotating part (61) and the second rotating part (62) to rotate to enable one of the first rotating part (61) and the second rotating part (62) to wrap the flexible strip (63) and to enable another one of the first rotating part (61) and the second rotating part (62) to release the flexible strip (63);
a vehicle body (65) connected to a middle part of the flexible strip (63); and
wheels (66) disposed on the rail (5) and rotationally connected to the vehicle body (65).

10. The non-contact health monitoring and physiotherapy equipment according to claim 9, wherein the vehicle body (65) comprises:
at least two sliding rods (651) disposed side by side, wherein:
the sliding rods (651) are perpendicular to the rail (5), and
the wheels (66) are rotationally connected to two ends of the sliding rods (651);
a connector (652) vertically connected to the sliding rods (651) to form a rectangular frame;
a slider (653) connected to the sliding rods (651) in a sliding manner and fastened to the monitoring module (1);
a rack (654) formed on one of the sliding rods (651) and extending along a length direction of the sliding rod (651);
a gear (655) rotationally connected to the slider (653) and meshed with the rack (654); and
a second motor (656) connected to the slider (653), wherein an execution shaft is coaxially connected to the gear (655).

11. The non-contact health monitoring and physiotherapy equipment according to claim 10, wherein:
the monitoring module (1) further comprises an angle detector (11), the angle detector (11) being connected to the displacement device in a communication manner and the angle detector (11) being configured to detect an elevation of a headboard;
the angle detector (11) comprises at least two distance sensors (111); and
detection ends of the distance sensors (111) are disposed upward and respectively aligned with two parts of the headboard with different heights.

12. The non-contact health monitoring and physiotherapy equipment according to claim 9, wherein:
the monitoring module (1) further comprises an angle detector (11), the angle detector (11) being connected to the displacement device in a communication manner and the angle detector (11) being configured to detect an elevation of a headboard;
the angle detector (11) comprises at least two distance sensors (111); and
detection ends of the distance sensors (111) are disposed upward and respectively aligned with two parts of the headboard with different heights.

13. The non-contact health monitoring and physiotherapy equipment according to claim 4, wherein:
the monitoring module (1) further comprises an angle detector (11), the angle detector (11) being connected to the displacement device in a communication manner and the angle detector (11) being configured to detect an elevation of a headboard;
the angle detector (11) comprises at least two distance sensors (111); and
detection ends of the distance sensors (111) are disposed upward and respectively aligned with two parts of the headboard with different heights.

14. The non-contact health monitoring and physiotherapy equipment according to claim 1, wherein:
the monitoring module (1) further comprises an angle detector (11), the angle detector (11) being connected to the displacement device in a communication manner and the angle detector (11) being configured to detect an elevation of a headboard;
the angle detector (11) comprises at least two distance sensors (111); and
detection ends of the distance sensors (111) are disposed upward and respectively aligned with two parts of the headboard with different heights.

15. A monitoring method of the non-contact health monitoring and physiotherapy equipment according to claim 1, comprising:
obtaining the limb position of the patient on the bed by the machine vision monitoring device;
obtaining the vital signs of a partial body of the patient by the monitoring module in the non-contact manner;
moving the bed or the monitoring module to enable the monitoring module to obtain vital signs of a whole body of the patient; and
outputting the vital signs of the whole body of the patient by the human-computer interaction terminal.

16. The monitoring method according to claim 15, wherein the displacement device comprises:
a walking device (2) configured to move on a ground, a wall, or a ceiling;
a linear driver (4) connected to an execution part of the walking device (2), wherein a driving direction of the linear driver (4) is vertical; and
an angular displacement device (3) connected to an execution part of the linear driver (4), wherein the angular displacement device (3) has at least one degree of freedom and the execution part of the angular displacement device (3) is connected to the monitoring module (1).

17. The monitoring method according to claim 15, wherein:
the displacement device comprises a rail (5) and a rail vehicle (6), wherein:
the rail (5) is mounted on a ground, a wall, or a ceiling and is suspended above the patient,
the rail vehicle (6) is connected to the rail (5) in a sliding manner, and
the monitoring module (1) is connected to the rail vehicle (6); and
the rail (5) comprises:
a first rail (51) disposed horizontally;
a second rail (52), wherein one end of the second rail (52) is rotationally connected to the first rail (51); and
a telescopic rod (53), wherein two ends of the telescopic rod (53) are respectively connected to free ends of the first rail (51) and the second rail (52).

18. The monitoring method according to claim 17, wherein:
the rail (5) further comprises a first limiting plate (54) and a second limiting plate (55) that are respectively disposed above the first rail (51) and the second rail (52);
the first rail (51) is parallel to the first limiting plate (54);
the second rail (52) is parallel to the second limiting plate (55); and
adjacent ends of the first limiting plate (54) and the second limiting plate (55) are hinged.

19. The monitoring method according to claim 18, wherein:
one end of the second rail (52) is hinged with one end of the second limiting plate (55) by a first connecting rod (56); and
another end of the second rail (52) is hinged with another end of the second limiting plate (55) by a second connecting rod (57).

20. A physiotherapy method of the non-contact health monitoring and physiotherapy equipment according to claim 1, comprising:
obtaining the limb position of the patient on the bed by the machine vision monitoring device;
obtaining the vital signs of a partial body of the patient by the monitoring module in the non-contact manner;
moving the bed or the monitoring module, to enable the monitoring module to obtain vital signs of a whole body of the patient, and obtain a part of the patient with an abnormal vital sign; and
providing a non-contact physiotherapy service to the part of the patient with an abnormal vital sign by the physiotherapy module.
